(19) United States
(12) Patent Application Publication
(10) Pub. No.: US 2021/0205206 A1
(43) Pub. Date: Jul. 8, 2021

(54) Wax for Hair Removal with Thermally Activated Pigment

(71) Applicant: American International Industries, a corporation, Los Angeles, CA (US)

(72) Inventors: Theresa Cooper, Los Angeles, CA (US); Euphrem Komras, Los Angeles, CA (US)

(73) Assignee: American International Industries, a corporation, Los Angeles, CA (US)

(21) Appl. No.: 17/068,614

(22) Filed: Oct. 12, 2020

Related U.S. Application Data
(63) Continuation-in-part of application No. 16/736,454, filed on Jan. 7, 2020, now Pat. No. 10,799,444.

(51) Int. Cl. A61K 8/92; A61K 8/02; A45D 26/00; A61Q 5/00

(52) U.S. Cl. CPC A61K 8/92; A61K 8/0208; A61Q 5/00; A45D 26/0019; A45D 26/0014

(57) ABSTRACT

A hot wax composition useful in removing hair from skin contains an effective amount of a thermally activated pigment which causes the composition to change color when it is heated sufficiently for use in hair removal and then change color again when the composition has cooled enough to be removed.

### CROSS REFERENCE TO RELATED APPLICATIONS

This application is a continuation-in-part application of U.S. Ser. No. 16/736,454, filed Jan. 7, 2020, the disclosure of which is specifically incorporated herein in its entirety.

### FIELD OF THE INVENTION

The present invention is in the field of wax used in cosmetic applications for hair removal.

### BACKGROUND OF THE INVENTION

It is known that wax can be heated for hair removal.

The present invention seeks to improve upon the existing art by offering a new and better wax for hair removal.

### SUMMARY OF THE INVENTION

The present invention is generally directed to a composition of matter and method of using it in which the hot wax formulation is heated up to an effective temperature for hair removal at which a thermally activated pigment in the composition changes color, at which point the composition can safely be applied to skin having hair to be removed, and then the thermally activated pigment changes color again when the composition has cooled sufficiently for the wax to be removed.

Accordingly, it is an object of the present invention to provide an improved hair removal wax.

This and further objects and advantages will be apparent to those skilled in the art in connection with the drawings and the detailed description of the invention set forth below.

### DETAILED DESCRIPTION OF THE INVENTION

In accordance with the present invention, a hair removal wax is formulated with a thermally activated pigment used to provide a visual indication of suitable use temperatures via changes in color. It is especially preferred that the thermally activated pigment change color when the formula has been heated to a temperature sufficient for use in hair removal (e.g., approximately 55 degrees Celsius) and then there is another change in color (e.g., return to the color prior to heating) when the formula has cooled back down to a temperature (e.g., room temperature) suitable for its removal from the skin.

In one especially preferred embodiment of the present invention, the thermally active pigment is visible when the composition is at room temperature, but the pigment color disappears when the composition is heated to approximately 55 degrees Celsius, but the pigment color reappears when the composition has cooled to room temperature. These color change properties provide a novel indication of suitability of use, not only in application of the hair removal wax, but also to its removal once it has been applied to a desired location from which hair is to be removed. Such application, and its reliance upon visual color changes for indicating desired temperatures, takes guess work out of heating and waiting for removal, and also helps to avoid overheating of the wax since its change in color upon heating indicates no further heating of the wax is required, which can help obviate the possibility of an overheated wax which can be too hot and thereby burn the skin.

In accordance with the present invention, and without intending to limit the present invention to the exact composition of the following formula, suitable hair removal wax can have the following composition (wherein the compositions are listed by weight percentages):

| | |
|---|---|
| Triethylene Glycol Hydrogenated Rosinate | 10.00-30.00% |
| (CAS#68648-53-3) | |
| Beeswax (Cera Alba, Cire d'abeille)(CAS#8012-89-3) | 5.00-25.00% |
| Ethylene/VA Copolymer (CAS#24937-78-08) | 1.00-5.00% |
| Glyceryl Rosinate (CAS#8050-31-5) | 50.00-80.00% |
| Paraffin (CAS#8002-74-2) | 1.00-5.00% |
| Stearalkonium Bentonite CAS#68953-58-2) | 1.00-5.00% |
| Thermal Activated Pigment (55C) | 0.50-2.00% |

The thermal activated pigment, in an especially preferred embodiment, is comprised of:

| | |
|---|---|
| Methoxypolyoxymethylene Melamine (CAS#68002-20-0) | 0.01-0.10% |
| 3-N-isoamyl-N-ethylamino-7, 8-benzofluoran | 0.02-0.20% |
| (CAS#115392-27-3) | |
| Stearyl Alcohol (CAS#112---92-5) | 0.45-1.70% |

It is believed that the weight percentages of the foregoing ingredients may be varied, while additional inert ingredients may be added, and equivalents to such ingredients may be substituted (in appropriate weight percentages), to get substantially the same result, as would be apparent to one skilled in the art exercising routine experimentation after studying the disclosure of the present invention.

With the foregoing in mind, the following additional formulations are within the scope of the present invention, and all the formulations are listed by weight formulations.

The first additional formulation has effective amounts of: (1) a thermally activated pigment that will change color at a preselected temperature suitable for use in application to a skin surface and a hot wax composition suitable for use in hair removal from the skin surface; (2) rosin and rosin derivatives that function for primary adhesion to hair; (3) plasticizers that function to increase flexibility of wax, for brittleness control and reduction; (4) synthetic adhesive film formers that function for secondary adhesion, cohesion and pliability control; (5) paraffin or other hydrocarbon or natural waxes that function as a binding agent, plasticizer and to provide lubrication; and (6) suspending agent that functions to suspend particles, pigments and colorants. In an especially preferred formulation, this formulation has the following composition:

| | |
|---|---|
| Thermally activated pigment | 0.5-2% |
| Rosin and rosin derivatives | 10-80% |
| Plasticizers | 1-20% |
| Synthetic Adhesive Film Formers | 1-10% |
| Paraffin or other hydrocarbon or natural waxes | 1-10% |
| Suspending Agent | 1-10% |

The second additional formulation has effective amounts of: (1) a thermally activated pigment that will change color at a preselected temperature suitable for use in application to a skin surface and a hot wax composition suitable for use in hair removal from the skin surface; (2) rosin and rosin derivatives that function for primary adhesion to hair; (3) plasticizers that function to increase flexibility of wax, for brittleness control and reduction; (4) synthetic adhesive film formers that function for secondary adhesion, cohesion and pliability control; (5) vegetable or mineral oils that function for lubrication, viscosity regulation, and emollient/skin protection; and (6) suspending agent that functions to suspend particles, pigments and colorants. In an especially preferred formulation, this formulation has the following composition:

| | |
|---|---|
| Thermally activated pigment | 0.5-2% |
| Rosin and rosin derivatives | 10-80% |
| Plasticizers | 1-20% |
| Synthetic Adhesive Film Formers | 1-10% |
| Vegetable or mineral oils | 1-10% |
| Suspending Agent | 1-10% |

The third additional formulation has effective amounts of: (1) a thermally activated pigment that will change color at a preselected temperature suitable for use in application to a skin surface and a hot wax composition suitable for use in hair removal from the skin surface; (2) synthetic adhesive film formers that function for primary adhesion, cohesion and pliability control; 3) plasticizers that function to increase flexibility of wax, for brittleness control and reduction; (4) vegetable or mineral oils that function for lubrication, viscosity regulation, and emollient/skin protection; and (5) suspending agent that functions to suspend particles, pigments and colorants. In an especially preferred formulation, this formulation has the following composition:

| | |
|---|---|
| Thermally activated pigment | 0.5-2% |
| Synthetic Adhesive Film Formers | 10-80% |
| Plasticizers | 1-20% |
| Vegetable or mineral oils | 1-10% |
| Suspending Agent | 1-10% |

Accordingly, it will be readily apparent to those skilled in the art that still further changes and modifications in the actual concepts described herein can readily be made without departing from the spirit and scope of the disclosed inventions.

What is claimed is:

1. A composition useful for hair removal, comprising:
(1) an effective amount of a thermally activated pigment that will change color at a preselected temperature suitable for use in application to a skin surface and a hot wax composition suitable for use in hair removal from the skin surface;
(2) an effective amount of at least one ingredient which promotes adhesion to hair selected from a first group consisting of rosin and rosin derivatives;
(3) an effective amount of plasticizer;
(4) an effective amount of synthetic adhesive film formers;
(5) an effective amount of at least one binding agent selected from a second group consisting of paraffin, hydrocarbon waxes and natural waxes; and
(6) an effective amount of a suspending agent.

2. The composition of claim 1, wherein the composition is comprised of:
(1) between 0.5-2 weight percent of the thermally activated pigment;
(2) between 10-80 weight percent of the first group;
(3) between 1-20 weight percent of plasticizers;
(4) between 1-10 weight percent of synthetic adhesive film formers;
(5) between 1-10 weight percent of the second group; and
(6) between 1-10 weight percent of the suspending agent.

3. A composition useful for hair removal, comprising:
(1) an effective amount of a thermally activated pigment that will change color at a preselected temperature suitable for use in application to a skin surface and a hot wax composition suitable for use in hair removal from the skin surface;
(2) an effective amount of at least one ingredient which promotes adhesion to hair selected from a first group consisting of rosin and rosin derivatives;
(3) an effective amount of plasticizer;
(4) an effective amount of synthetic adhesive film formers;
(5) an effective amount of at least one binding agent selected from a second group consisting of vegetable and mineral oils; and
(6) an effective amount of a suspending agent.

4. The composition of claim 3, wherein the composition is comprised of:
(1) between 0.5-2 weight percent of the thermally activated pigment;
(2) between 10-80 weight percent of the first group;
(3) between 1-20 weight percent of plasticizers;
(4) between 1-10 weight percent of synthetic adhesive film formers;
(5) between 1-10 weight percent of the second group; and
(6) between 1-10 weight percent of the suspending agent.

5. A composition useful for hair removal, comprising:
(1) an effective amount of a thermally activated pigment that will change color at a preselected temperature suitable for use in application to a skin surface and a hot wax composition suitable for use in hair removal from the skin surface;
(2) an effective amount of synthetic adhesive film formers;
(3) an effective amount of plasticizers;
(4) an effective amount of at least one ingredient selected from the group consisting of vegetable and mineral oils; and
(5) an effective amount of a suspending agent.

6. The composition of claim 5, wherein the composition is comprised of:
(1) between 0.5-2% weight percent of the thermally activated pigment;
(2) between 10-80 weight percent of the synthetic adhesive film formers;
(3) between 1-20 weight percent of plasticizers;
(4) between 1-10 weight percent of the first group; and
(5) between 1-10 weight percent of the suspending agent.
